(19) United States
(12) Patent Application Publication
(10) Pub. No.: US 2013/0131601 A1
(43) Pub. Date: May 23, 2013

(54) METHOD AND SYSTEM FOR DIFFERENTIATING DRUG DELIVERY DEVICES

(71) Applicants: Christian Pommereau, Frankfurt (DE); Anke Liewald, Frankfurt (DE)

(72) Inventors: Christian Pommereau, Frankfurt (DE); Anke Liewald, Frankfurt (DE)

(73) Assignee: SANOFI-AVENTIS DEUTSCHLAND GMBH, Frankfurt am Main (DE)

(21) Appl. No.: 13/639,410

(22) PCT Filed: Apr. 7, 2011
(86) PCT No.: PCT/EP2011/055396

§ 371 Date: Jan. 18, 2013

Related U.S. Application Data
(60) Provisional application No. 61/322,700, filed on Apr. 9, 2010.

(30) Foreign Application Priority Data
Jul. 29, 2010 (EP) 10171161.2

(51) Int. Cl. A61M 5/31

(52) U.S. Cl. CPC A61M 5/3129
USPC 604/189

(57) ABSTRACT

A method and system is disclosed for differentiating a drug delivery device that may be used to administer a plurality of drug types. The drug delivery system includes a drug delivery device and a differentiation shell. The drug delivery device holds a given medicament. A differentiation shell is attachable to the drug delivery device. The differentiation shell may include at least one shell-type feature configured to attach to a drug delivery device and at least one differentiation feature. The differentiation feature identifies the given medicament in the drug delivery device.

### CROSS REFERENCE TO RELATED APPLICATIONS

The present application is a U.S. National Phase Application pursuant to 35 U.S.C. §371 of International Application No. PCT/EP2011/055396 filed Apr. 7, 2011, which claims priority to U.S. Provisional Patent Application No. 61/322,700 filed Apr. 9, 2010 and European Patent Application No. 10171161.2 filed Jul. 29, 2010. The entire disclosure contents of these applications are herewith incorporated by reference into the present application.

### FIELD OF INVENTION

The present disclosure is generally directed to drug delivery devices. More particularly, the present disclosure is generally directed to drug delivery devices, for example injection devices, that may be filled with different drugs or different drug concentrations. Exemplary medical delivery devices include, but are not limited to syringes, pen type injection syringes, pumps, inhalers, or other similar injection or infusing devices. The medical delivery devices may require at least one reservoir containing at least one medicament.

### BACKGROUND

Medicament reservoirs such as ampoules, cartridges, or vials are generally known. Such reservoirs may be especially used for medicaments that may be self administered by a patient. For example, with respect to insulin, a patient suffering from diabetes may require a certain amount of insulin to either be injected via a pen type injection syringe or infused via a pump. With respect to certain known reusable pen type drug delivery devices, a patient loads a cartridge containing the insulin into a proximal end of a cartridge holder. After the cartridge has been correctly loaded, the user may then be called upon to select a dose of medicament. Multiple doses may be dosed from the cartridge. Further, reservoirs containing different medicaments may be loaded into the cartridge holder. Thus, a given drug delivery device may be used to deliver a plurality of different medicaments. For example, a given drug delivery may be used to deliver short acting insulin in a given scenario and long acting insulin in another scenario. As another example, a first drug delivery device of a certain type may be used to deliver a first medicament, and a second drug delivery device of the same type may be used to deliver a second medicament different than the first.

Where the drug delivery device comprises a reusable device, once the loaded cartridge is empty, the cartridge holder may be disconnected from the drug delivery device and the empty cartridge may be removed and replaced with a new cartridge. Most suppliers of such cartridges recommend that the user dispose of the empty cartridges properly. Where the drug delivery device comprises a disposable device, once the cartridge is empty, the user may be recommended to dispose of the entire device.

Such known self administration systems that allow a given drug delivery device to deliver a plurality of different medicaments may have certain limitations. For example, since a given drug delivery device may be used to deliver a plurality of medicaments, differentiation and cartridge cross-use issues may arise. As an example, a user of a given drug delivery device may typically use a given drug delivery device, in particular a first drug delivery device, to deliver a first medicament (e.g., long acting insulin). However, under a scenario, a second drug delivery device that is the same type of the first device or a similar device as the given first drug delivery device may contain a second medicament (e.g., a short acting insulin). A user may use this second device, expecting that the second drug delivery device contains the first medicament because the user associates the first medicament with the given drug delivery device. The user may then inject the second medicament when the user is expecting a dose of the first medicament.

Avoiding such a situation may be quite important, since the administration of a potentially incorrect dose of a medicament such as a short acting insulin in lieu of a long insulin could result in injury or even death. There may be, therefore, a need to differentiate drug delivery devices that may be used to deliver a plurality of medicaments.

The general problem to be solved by the present invention is to provide a drug delivery system where the security for the user is improved.

### SUMMARY

In one aspect, there is provided a differentiation shell attachable to a drug delivery device. The differentiation shell may comprise at least one shell-type feature. The shell-type feature comprises the shape of a shell. In particular, the shell-type feature may have a certain amount of rigidity. Thereby, the shape may be basically the same before the shell is attached to the device and after the shell is attached to the device. The shell may comprise a curved shape. As examples, the shell-type feature may comprise the shape of a sleeve or the shape of parts of a sleeve. In particular, the shell-type feature may comprise the shape of a cylindrical sleeve, for example, a circular cylindrical sleeve, or parts thereof. The shape of the shell-type feature may be adapted to the shape of the drug delivery device.

Moreover, the differentiation shell may comprise at least one differentiation feature, wherein the differentiation feature may be suitable for identifying a feature of the drug delivery device or of a medicament used with the drug delivery device, for example held in the drug delivery device. As examples, the differentiation feature may comprise at least one of a tactile differentiation feature, an optical differentiation feature, for example, the color of emitted light, or an electronic differentiation feature. An optical differentiation feature may comprise a light-emitting material, for example a light-emitting plastic. The differentiation shell may comprise one or several ergonomic features for improving the handling of the device. The ergonomic features may also serve as differentiation features.

The differentiation shell may comprise a single shell-type feature or a plurality of shell-type features. As an example, a first shell-type feature may comprise first connection means and a second shell-type feature may comprise second connection means. The first and second connection means may be configured to interact with each other such that the first and second shell-type features are enabled to be connected together.

According to a specific embodiment, a differentiation shell is attachable to a drug delivery device and comprises at least one shell-type feature configured to attach to the drug delivery device and at least one differentiation feature, wherein the differentiation feature identifies a given medicament.

In a further aspect, there is provided a set of at least two differentiation shells as described above, wherein the differentiation shells comprise differentiation features which enable a differentiation from each other. The differentiation shells may be attached to drug delivery devices. In particular, one of the differentiation shells may be attached to a first drug delivery device and a second differentiation shell may be attached to a second drug delivery device. The first and the second drug delivery device may be similar or identical and may comprise different medicaments. When the differentiation shells are attached to the devices, the differentiation features may enable a user to identify the medicaments contained in the devices or otherwise to distinguish between the devices.

In a further aspect, there is provided a drug delivery system comprising a drug delivery device and a differentiation shell attachable to the drug delivery device. As an example, the drug delivery device may be an injection device, in particular a pen-type injection device. The differentiation shell may have any of the features as described above. The differentiation shell may be used for identifying the drug delivery device, a feature of the device or a medicament used with, for example held within, the device. The drug delivery device may be used to administer a plurality of drug types. For example, a first drug delivery device of a given type may be used for delivering a first medicament, for example, short acting insulin, and a second drug delivery device of the same given type may be used to deliver a second medicament, for example, long acting insulin. Various other medicaments are possible as well. Beneficially, the differentiation shell having the differentiation feature may serve to differentiate the given drug delivery device from other drug delivery devices of the same or of a similar type that may hold a medicament different from the given medicament.

The differentiation shell may be placed around a portion of a drug delivery device, for example a body portion of the drug delivery device. In this case, the shape of the shell-type feature may correspond to the outer contour of the body portion of the drug delivery device. As an example, the drug delivery device may be a pen-type device. Here, the body portion may have a cylindrical outer contour. In this case, the shell-type feature may comprise the shape of a hollow cylindrical sleeve. The differentiation shell may be configured to be wrapped around the body portion of the drug delivery device.

In one embodiment, the differentiation shell may be attached by mechanical means to the drug delivery device, for example, attached by snap-fitting means to the device. In particular, the differentiation shell may comprise first snap-fitting elements configured to interact with second snap-fitting elements of the device.

Furthermore, in one embodiment, the drug delivery device may be configured such that the drug delivery device is unable to deliver a dose when a differentiation shell is not attached to the drug delivery device or when a correct differentiation shell is not attached to the device. Thereby, users may be prevented from using a device that is holding an incorrect medicament, for example, injecting a short acting insulin when the user is expecting long acting insulin. As examples, the drug delivery device may comprise a lock feature, for example at least one of a mechanical lock feature and an electronic lock feature. The lock feature may be configured such that the device is disabled from delivering a drug in a locked state of the lock feature. The differentiation shell may be configured to unlock the lock feature when the differentiation shell is attached to the drug delivery device.

Moreover, the drug delivery system may comprise a radio-frequency identification (RFID) device, wherein the RFID device may provide information to the drug delivery device or the drug delivery device may provide information to the RFID device. As an example, the RFID device may comprise information specific to a certain user and provide the information, for example drug-related information, to the drug delivery device. In one embodiment, the RFID device provides or receives information from the differentiation shell, in particular provides or receives information from an electronic differentiation feature of the differentiation shell. Thereby, the RFID device may be provided with information regarding the medicament contained in the device. In turn, the RFID device may send information to the electronic differentiation feature or to further electronic means of the device which, for example, may affect dose setting or dose delivery procedures. As examples, the information provided to the device may comprise a maximum dose for the medicament, a minimum dose for the medicament, a required dose for the medicament or a dosing speed for the medicament.

According to a specific embodiment, a drug delivery system comprises a drug delivery device, wherein the drug delivery device holds a given medicament; a differentiation shell attachable to the drug delivery device, the differentiation shell comprising: at least one shell-type feature configured to attach to a drug delivery device; and at least one differentiation feature, wherein the differentiation feature identifies the given medicament.

The term “drug” or “medicament”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
   wherein the group -Lys6-NH2 may be bound to the C-terminus of the Exendin-4 derivative;
   or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
   or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The scope of the invention is defined by the content of the claims. The invention is not limited to specific embodiments but comprises any combination of elements of different embodiments. Moreover, the invention comprises any combination of claims and any combination of features disclosed by the claims.

These as well as other advantages of various aspects of the present disclosure will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1A illustrates an exemplary pen type drug delivery device;

FIG. 1B illustrates an exemplary drug cartridge;

FIG. 2 illustrates an exemplary drug delivery system;

FIG. 3 illustrates an exemplary drug delivery system; and

FIG. 4A and 4B illustrate a user holding the exemplary drug delivery system of FIG. 3.

### DETAILED DESCRIPTION

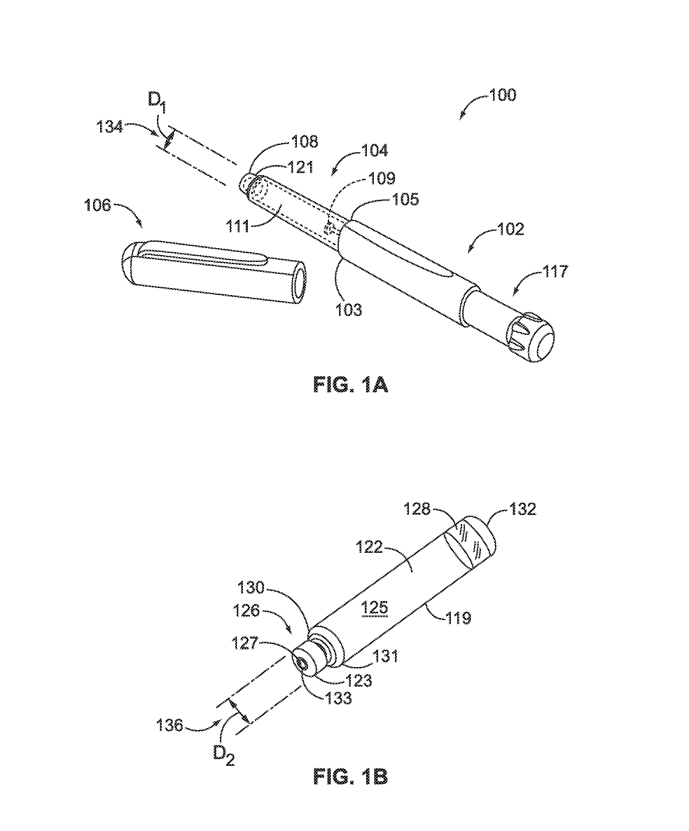

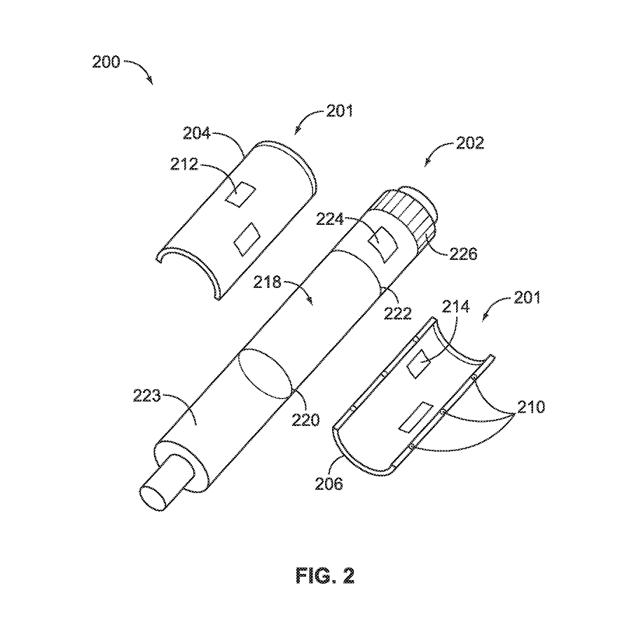

Referring to FIG. 1A, there is shown a drug delivery device 100 in the form of a pen type syringe. This drug delivery device 100 comprises a dose setting mechanism 102, a cartridge holder 104, and a removable cap 106. A proximal end 105 of the cartridge holder 104 and a distal end 103 of the dose setting mechanism 102 are removably secured together. The dose setting mechanism 102 comprises a piston rod 109, such as a threaded piston rod 109 that rotates when a dose is injected.

To inject a previously set dose, a double ended needle assembly is attached to a distal end 108 of the cartridge holder 104. Preferably, the distal end 108 of the holder 104 comprises a thread 121 (or other suitable connecting mechanism such as a snap lock, snap fit, form fit, or bayonet lock mechanism) so that the needle assembly may be removably attached to the distal end 108 of the holder 104. When the drug delivery device 100 is not in use, the removable cap 106 can be releasably retained over the cartridge holder 104.

An inner cartridge cavity 111 defined by the cartridge holder 104 is dimensioned and configured to securely receive and retain a cartridge, such as glass cartridge. FIG. 1B illustrates a perspective view of the cartridge 119 that may be used with the drug delivery device 100 illustrated in FIG. 1A. Typically, the cartridge 119 is manufactured of glass and includes a generally tubular barrel 122 extending from a distal end 130 to a proximal end 132.

At the distal end 130, the cartridge 119 includes a smaller diameter neck 126 and this neck projects distally from the shoulder 131 of the barrel 122. Preferably, this smaller diameter neck 126 is provided with a large diameter annular bead 123 and this bead 123 extends circumferentially thereabout at the extreme distal end of the neck 126 and defines an opening 127. A pierceable seal or septum 133 is securely held across the opening 127 by a metallic sleeve or a ferrule.

The medicament 125 is pre-filled into the cartridge 119 and is retained within this cartridge 119, in part, by the pierceable seal 133, a ferrule, and the stopper 128. The stopper 128 is in sliding fluid-tight engagement with the inner tubular wall of the barrel 122. Axially directed forces acting upon the stopper 128 during dose injection or dose administration urges the medication 125 from the cartridge 119 though a double ended needle mounted onto the distal end 130 of the cartridge holder 104 and into the injection site. Such axially directed forces may be provided by the piston rod 109 working in unison with the dose setting member 102.

A portion of the cartridge holder 104 defining the cartridge holder cavity 111 is of substantially uniform diameter represented in FIG. 1A by D₁ 134. This diameter D₁ 134 is preferably slightly greater than the diameter D₂ 136 of the cartridge 119 as shown in FIG. 1B. The interior of the cartridge holder 104 includes an inwardly-extending annular portion or stop that is dimensioned to prevent the cartridge 119 from moving within the cartridge holder 104. In this manner, when the cartridge 119 is loaded into the cavity 111 of the cartridge holder 104 and the cartridge holder 104 is then connected to the dose setting member 102, the cartridge assembly 119 will be held within the cartridge cavity 111.

A number of doses of a medicament 125 may be dispensed from the cartridge 119. Preferably, the cartridge 119 contains a type of medicament 125 that must be administered often, such as one or more times a day. One such medicament 125 is insulin.

The dose setting mechanism 102 comprises a dose setter 117 at the proximal end of the dose setting mechanism 102. In one preferred arrangement, the dose setter 117 is rotated to set a dose. To administer this set dose, the user attaches the needle assembly comprising a double ended needle on the distal end of the cartridge holder 104. In this manner, the needle assembly pierces the seal 133 of the cartridge 119 and is therefore in liquid communication with the medicament 125. The user pushes on the dose setter 117 to inject the set dose. The same dose setting and dose administration procedure is followed until the medicament 125 in the cartridge 119 is expended and then a new cartridge 119 must be loaded in the device.

A drug delivery device, such as drug delivery device 100, may be used to administer a plurality of different drug types (i.e., medicaments). For example, a first drug delivery device of a given type may be used to deliver a first medicament 125 (e.g., short acting insulin), and a second drug delivery device of the same given type may be used to deliver a second medicament 125 (e.g., long acting insulin). Various other medicaments 125 are possible as well. Since the same or similar type of drug delivery device 100 may be used to deliver different drugs, differentiation issues between the same or similar drug delivery devices 100 that hold different medicaments 125 are likely to arise. For instance, mix-up issues between devices 100 having different medicaments 125 are quite possible. A patient using a drug delivery device 100 of that type may expect one medicament, but may unknowingly inject the wrong medicament. As is known in the art, injecting the wrong medicament may cause serious medical issues. Thus, there is a need to better differentiate drug delivery devices 100 that may be used to administer a plurality of drug types.

In accordance with embodiments of the disclosed concept, a differentiation shell may be used to differentiate drug delivery devices such as injection syringes or similar drug delivery devices that may be filled with different drugs. In particular, the differentiation shell may be used with the drug delivery device 100 as described in FIGS. 1A and 1B. For example, a first differentiation shell may be used to identify a first drug (e.g., a long acting insulin) and a second differentiation shell may be used to identify a second drug (e.g., a short acting insulin). Both drugs may be contained in cartridges 119 as shown in FIG. 1B.

A drug delivery system according to an embodiment includes a drug delivery device and a differentiation shell attachable to the drug delivery device. The drug delivery device holds a given medicament 125 or is suitable for holding a medicament 125. The differentiation shell comprises at least one shell-type feature configured to attach to a drug delivery device and at least one differentiation feature 212, wherein the differentiation feature 212 identifies the given medicament 125. The shells may comprise various features that serve to differentiate drugs and/or devices. These features will be described in greater detail below.

According to an example, a differentiation shell may be placed around a portion of a drug delivery device. In a preferred embodiment, the differentiation shell is placed around a center-body portion 312 of the drug delivery device. Further, in an embodiment, a drug delivery device will not operate properly unless a correct differentiation shell is attached to the drug delivery device. For example, the drug delivery device may be configured such that the drug delivery device is unable to deliver a dose when a differentiation shell is not attached to the drug delivery device or when a correct differentiation shell is not attached to the device. Beneficially, by not operating unless a differentiation shell or unless a correct differentiation shell is attached to the device, users may be prevented from using a device that is holding an incorrect medicament (e.g., injecting a short acting insulin when the user is expecting long acting insulin).

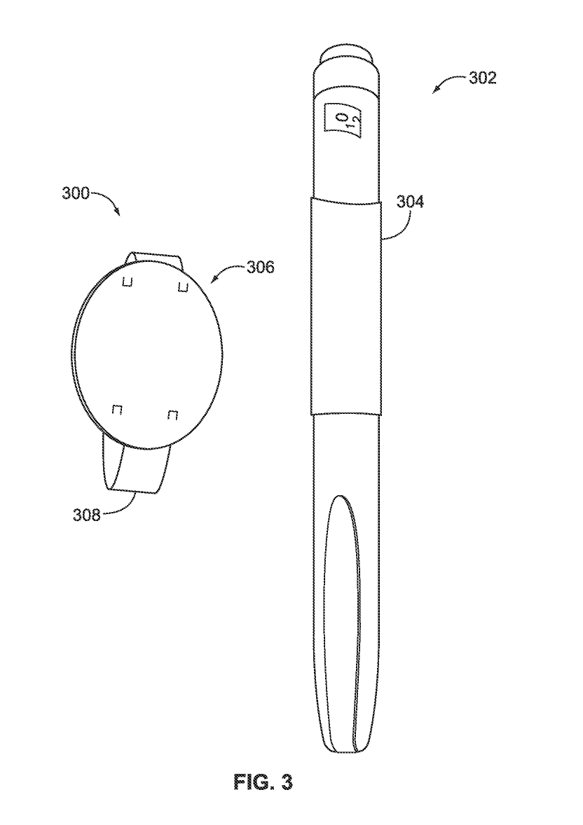

An example drug delivery system 200 is shown in FIG. 2. Drug delivery system 200 includes differentiation shell 201 and drug delivery device 202. Differentiation shell 201 may be assembled onto drug delivery device 202. In this particular example, the differentiation shell 201 includes two shell-type features, i.e. half shell 204 and half shell 206. The shell-type features have shapes of partial sleeves. Half shell 204 includes connection features (not shown here) and half shell 206 includes connection features 210. These connections features may take a variety of forms. In this example, connection features are male and female snap-fit connection features. In particular, half shell 206 includes female connection features 210 and half shell 204 includes male connection features (not shown).

The differentiation shell 201 also includes a differentiation feature, such as differentiation feature 212. The differentiation feature 212 may identify the given medicament that is in the particular drug delivery device 202. For example, if the given medicament is long acting insulin, the differentiation feature 212 may identify that the device holds long acting insulin. Alternatively, if the given medicament in drug delivery device 202 is short acting insulin, the differentiation feature 212 may identify that the device holds short acting insulin. The differentiation shell 212 may also include, for example, a cut-out 214. Such a cut-out 214 may provide visibility for a data-matrix code or other relevant security information. The data matrix code may be used to identify the drug contained within the drug delivery device. As an example, the data matrix code may be located on a part, for example on a center body portion 218, of the drug delivery device 202 where the differentiation shell 201 is attached to and may be visible from the outside through the cutout 214. Further, the differentiation shell may include other features, such as additional safety features and/or ergonomic features, which will be discussed in more detail below.

In the example of FIG. 2, the differentiation shell 201 is designed to fit around the center body portion 218 of the drug delivery device 202. Specifically, in this example, the central body portion 218 is a part of the body or the body of the device 202 and may lie between (i) the interface 220 between the cartridge holder 104 and dose setting mechanism 102 of the device and (ii) the point 222 that lies below, for example near the distal end of, the dose screen 224, through which a user may see the units of a dialed dose. However, it should be understood that in other embodiments, a differentiation shell may cover other portions of the drug delivery device 202. For example, a differentiation shell may cover the cartridge holder portion 223. In another example, the differentiation shell may cover the part of the device 202 from point 222 to the dose dial 226. In this example, the differentiation shell may have a cut-out or a see-through portion (e.g., glass or clear plastic) that allows a user to see the dose screen 224. Other examples are possible as well.

The at least one differentiation feature 212 in the differentiation shell 201 may take a variety of forms. Further, the location of differentiation feature 212 is intended as an example only. A differentiation feature 212 or features may be located anywhere of the differentiation shell 201. Further, the differentiation shell itself may serve as the differentiation feature 212. In an embodiment, differentiation shells may be designed so that differentiation shells of different colors are intended to identify different drugs. In an example, shells may be composed of or may include light-emitting plastics, such as light-emitting plastic materials from Lanxess (LISA). The differentiation feature 212 may be the color of light emitted from the light-emitting plastic material. Different colors may signify or identify different types of drugs. For example, the color red may be used to signify or identify short acting insulin and the color green may be used to identify long acting insulin. Other examples are possible as well.

In another embodiment, the at least one differentiation feature of the differentiation shell may include a tactile differentiation means (e.g., a tactile differentiation element or elements). Exemplary tactile differentiation means could include Braille letters, geometric features, or an arrangement of hard/soft material combinations, and/or plastic/metal or plastic/fabric combinations. Such tactile differentiation means may be of particular benefit for visually-impaired users.

In yet another embodiment, the differentiation shell may have ergonomic features. Ergonomic features may be used to further differentiate drug delivery devices. For example, differentiating ergonomic features may be integrated into the shells to distinguish the shells for left-handed users and right-handed users. Ergonomic features may beneficially make a drug delivery device easier to use, which may be of particular benefit for patients suffering from arthritis. Further, ergonomic features may also be particularly beneficial for visually impaired users. For example, a visually impaired left-handed user may know not to use a drug delivery that includes an ergonomic feature that is designed for a right handed patient.

Other safety features may beneficially be integrated into the disclosed differentiation shells. For example, the shells may have integrated magnifying elements (e.g., a lens). These magnifying elements may serve to magnify certain elements of the drug cartridge, the cartridge holder, the drug delivery device or the differentiation shell itself, such as an element showing what drug is contained in the drug delivery device. For example, such a magnifying element or elements may be used to magnify dose numbers that are shown on a display or that may printed along a dialing element. Such a magnifying element may be placed on top of numbers to be magnified or with movable elements along the labels to enable magnifying of expiration date or any other safety relevant information provided by labels or other appropriate means. These magnifying elements may be of particular benefit for visually-impaired users. Additional safety features that may be included on the differentiation shell include a vocal warning, a flashing light, an alarm noise, or perhaps a vibration indication.

The differentiation shell 201 may be assembled on the drug delivery device 202 by an educated user prior to use of the drug delivery device 202. For example, an educated patient, nurse, or doctor may assemble the differentiation shell 201 on the drug delivery device 202. Assembly by an educated user may beneficially ensure that only correct differentiation shells are assembled on correct drug delivery devices. For example, a drug delivery device having a given medicament (e.g., short acting insulin) can reject a differentiation shell that is intended to identify a different medicament (e.g., long acting insulin). This can occur by providing the drug delivery device with an electronic coding circuit of the shell/device pair and appropriate warning message to the patient by way of RFID technology having a transponder and transceiver combination.

In an embodiment, mechanical coding of the differentiation shells connection features (e.g., snap features) onto the body of the drug delivery device may avoid inadvertent cross use situations. For example, male snap features on either the first or the second shell portions may not engage into female snaps features on the other shell portion.

In this example of FIG. 2, the differentiation shell 201 includes the shell-type features in the form of two half components 204, 206 that each cover approximately 180 degrees of a portion of the circumference of the drug delivery device 202. When combined, the shells cover a portion of the device 202 around the entire 360 degree circumference of the device 202. However, it should be understood that a differentiation shell in accordance with the disclosed concept may include shell-type features, for example, shells, that cover differing degrees of the circumference of the device. For instance, a differentiation shell may cover 180 degrees around the drug delivery device, 240 degrees around the drug delivery device, or 300 degrees around the drug delivery device. Other examples are possible as well.

Further, a differentiation shell may include more than two components. For instance, the differentiation shell may comprise three shell-type features that connect together to form a complete shell around the device. Other examples are possible as well. However, it should be noted that fewer components, such as the two components 204, 206 shown in FIG. 2, may be preferred because fewer components allow for an easier assembly process by the patient or by healthcare professionals.

Still further, although the half shells of FIG. 2 are depicted as separate pieces, the half shells could be connected together before the assembly with the device. For example, the half shells may be connected together by a hinge. In particular, the half shells could be unreleasably connected together before the assembly with the device. Such an embodiment may simplify the assembly process of attaching a differentiation shell to the drug delivery device.

As mentioned above, in an embodiment, the drug delivery device may not operate properly unless a correct differentiation shell is attached to the drug delivery device. For instance, the drug delivery device may have a lock feature that is capable of a locked position and an unlocked position. If the lock feature is in the locked position, the lock may prevent the drug delivery device from at least one of dialing a dose and dispensing a dose. In an example, the lock feature is a mechanical lock. When a correct differentiation shell is attached to the device, the correct shell may deactivate the mechanical lock and move it into an unlocked position. Therefore, attaching this correct differentiation shell may allow a user to use the drug delivery device. However, if an incorrect shell or no differentiation shell is attached to the device, the mechanical lock feature will remain locked, thereby preventing use of the device. In another example, the drug delivery device may include an electronic lock feature. Other lock features are possible as well.

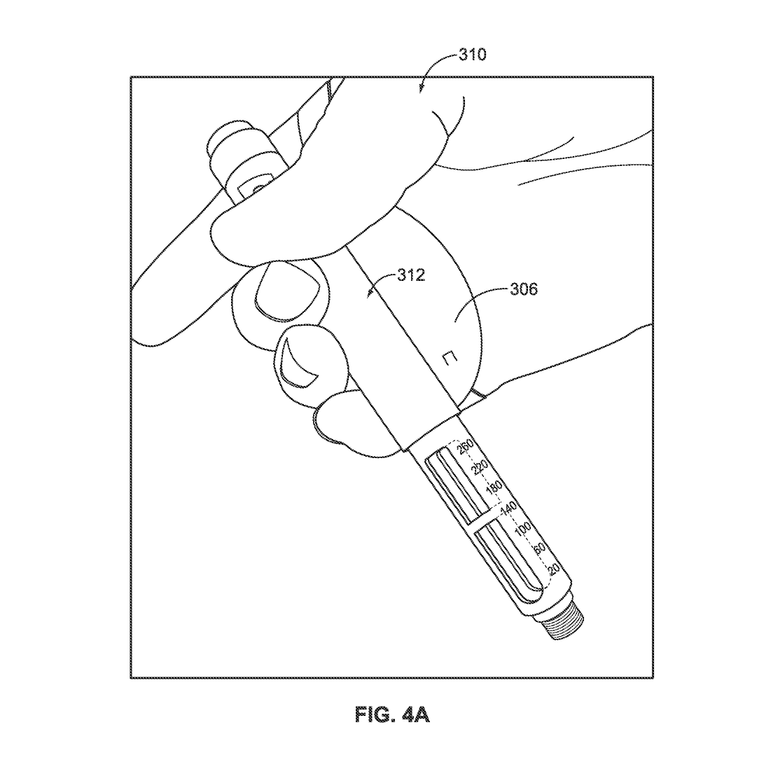

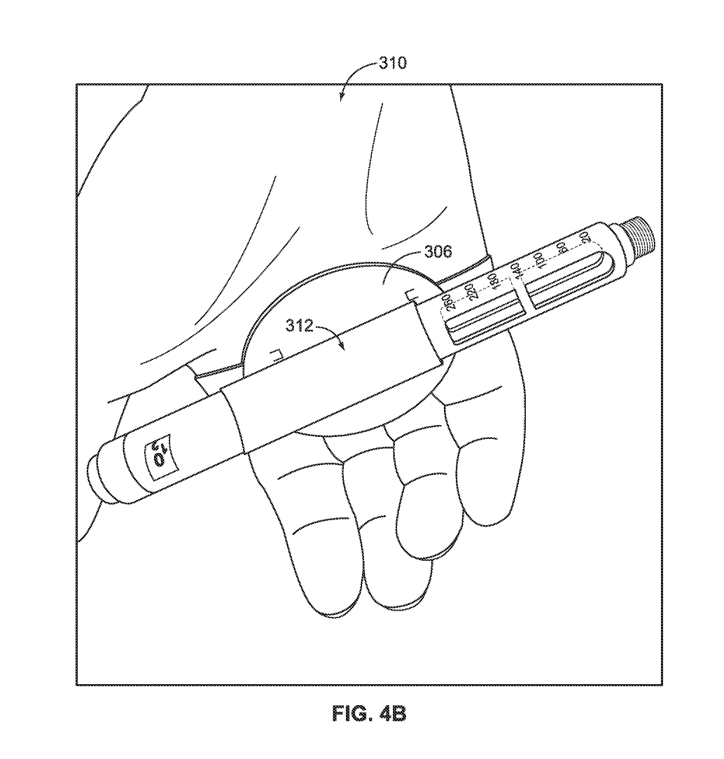

A drug delivery system in accordance with embodiments of the disclosed concept may also include a radio-frequency identification (RFID) device. FIG. 3, 4A, and 4B depict a drug delivery system 300. Specifically, FIG. 3 depicts a perspective view of the drug delivery system 300, and FIGS. 4A-4B depict the drug delivery system 300 in the hand 310 of a user.

Referring to FIG. 3, the drug delivery system 300 includes drug delivery device 302, differentiation shell 304, and RFID device 306. Using RFID technology, the RFID device 306 can provide information to the drug delivery device 302 and vice versa. In particular, the differentiation shell 304 may comprise an electronic differentiation feature which provides information to the RFID device 306 or vice versa. The RFID device 306 in this example includes a palm strap 308, which a user may use to strap the RFID device 306 around the user's palm. For example, FIGS. 4A and 4B show RFID device 306 strapped around the hand 310 of a user. In this example, the RFID device 306 contacts the center-body portion 312 of the drug delivery device 302 when the user holds the device 302 in his or her hand. Other methods of holding the RFID device 306 or attaching the RFID device 306 to a user are possible as well.

In an example, the RFID device 306 is an RFID device 306 of a given patient, and the information provided to the drug delivery device 302 is drug-related information particular to the given patient. In other words, an RFID device 306 may be specifically tailored or programmed for a given patient. For example, the information may be a maximum dose for the given medicament 125 that a user may receive, a minimum dose for the given medicament that the user needs, a required dose for the given medicament for the user, and a dosing speed for the given medicament. Other examples are possible as well. For instance, the RFID device 306 may include information that indicates drug types a user may receive and drug types a user may not receive. The RFID device 306 may also include information related to a specific dosing regime for the user. Given such a device, an RFID device 306 may facilitate proper use of the drug delivery device 302. The RFID device 306 may further include a display that displays such information to the user.

Further, if a user holding an RFID device 306 attempts to use a drug delivery device 302 holding a drug that the user may not receive, the drug delivery device 302 may be configured to prevent the user from injecting a dose. For instance, the drug delivery device 302 may include an electronic latch that is activated when the device 302 recognizes (based on the information from the RFID device 306) that the particular user cannot receive the medicament in the device 302. Further, when a user attempts to use a drug delivery device 302 containing an incorrect drug, the display of the RFID device 306 may indicate such information. Alternatively, the drug delivery device 302 may include a display feature that indicates such information. In an embodiment, the differentiation shell may include such a display.

In another example, the drug delivery device 302 may provide information to the RFID device 306. For example, the drug delivery device may provide information such as the medicament in the device, a number of doses dispelled from the device and a remaining amount of the given medicament in the device. In addition, this information could include drug expiration data, temperature of drug, time and date of last injection, a warning for a missed safety shot, a warning to the user to change a needle, name and/or location of the manufacturer, brand identification of the drug and a warning instruction for proper use of the device, or a warning that the user is approaching a last dose of the cartridge contained within the drug delivery device. The said information may also help the user to identify counterfeit medicament.

The differentiation shells 304 and RFID devices 306 in accordance with embodiments of the disclosed concepts may be used in conjunction with currently known drug delivery devices or drug delivery devices developed in the future.

Although aimed primarily at the insulin market, the disclosed concepts may apply to other drugs. The concept may apply to various devices, including the following examples:

a. An injector pen with a cartridge (e.g., 3 ml cylindrical glass cartridge) non-removably retained in a holder, so that the holder will be disposed of with the primary pack.

b. Any drug delivery device, with any type of primary pack, e.g. inhaler, pouch.

Although described primarily with reference to a cartridge 119 assembly that attaches to an injection pen type drug delivery device, the disclosed embodiments may apply to any drug delivery device, with any type of reservoir or primary pack, e.g. inhaler, pouch. The differentiation shells may be adjusted to work with differently shaped devices and differently shaped reservoirs.

The disclosed embodiments for differentiating drug delivery devices result in a number of advantages. For example, the differentiating shells may serve to beneficially alert a user of what drug a particular drug delivery device is holding, when the drug delivery device may be used to administer a plurality of drug types. Further, by preventing operating when a differentiation shell is not attached to a device or when a user is not holding an RFID device 306, the disclosed concepts may beneficially prevent a user from injecting a wrong medicament 125. The differentiation shells and RFID devices 306 facilitate proper use of a drug delivery device that may be used to administer a plurality of different drug types. Thus, differentiation and mix-up issues are reduced or prevented with the disclosed differentiation tactics.

Exemplary embodiments of the present invention have been described. However, as those of skill in the art will recognize certain changes or modifications to such arrangements may be made. Those skilled in the art will understand, however, that further changes, modifications, revisions and/or additions may be made to the presently disclosed arrangements without departing from the true scope and spirit of the present invention, which is defined by the claims.

1-14. (canceled)

15. A drug delivery system comprising:
a drug delivery device, wherein the drug delivery device is suitable for holding a medicament;
a differentiation shell comprising:
at least one shell-type feature comprising the shape of a shell and being configured to be attached to the drug delivery device; and
at least one differentiation feature, wherein the differentiation feature is suitable for identifying a feature of the drug delivery device or of a medicament used with the drug delivery device,
the differentiation shell being attachable to the drug delivery device and the differentiation feature being suitable for identifying a feature of the drug delivery device or of a medicament used with the drug delivery device,
wherein the drug delivery device is configured such that the drug delivery device is unable to deliver a dose when the differentiation shell is not attached to the drug delivery device.

16. The drug delivery system according to claim 15, wherein the at least one shell-type feature comprises the shape of a sleeve or of parts of a sleeve.

17. The drug delivery system of claim 15, wherein the at least one shell-type feature comprises:
a first shell-type feature having a first connection means; and
a second shell-type feature having a second connection means, wherein the first and second connection means are configured to interact with each other such that the first and second shell-type features are enabled to be connected together.

18. The drug delivery system of claim 17, wherein the first connection means comprises a female connection means and the second connection means comprises a male connection means.

19. The drug delivery system of claim 15, wherein the at least one shell-type feature comprises a light-emitting material, wherein the at least one differentiation feature comprises light emitted from the light-emitting material.

20. The drug delivery system of claim 15, comprising one or several ergonomic features for improving the handling of the differentiation shell.

21. The drug delivery system of claim 15, wherein the at least one differentiation feature comprises a tactile differentiation feature.

22. The drug delivery system of claim 15, comprising a magnifying feature, wherein the magnifying feature is configured to magnify at least one of a portion of the differentiation shell and a portion of the drug delivery device.

23. The drug delivery system of claim 15, wherein the drug delivery device comprises a lock feature configured to disable the drug delivery device (100, 202, 302) from delivering a medicament in a locked state of the lock feature and wherein the differentiation shell is configured to unlock the lock feature when the differentiation shell is attached to the drug delivery device.

24. The drug delivery system of claim 23, wherein the lock feature comprises at least one of a mechanical lock feature and an electronic lock feature.

25. The drug delivery system of claim 15, further comprising an RFID device, wherein the RFID device provides information to the drug delivery device or the drug delivery device provides information to the RFID device.

26. The drug delivery system of claim 25, wherein the information is drug-related information particular to a specific user.

27. The drug delivery system of 25, wherein the RFID device provides information to the drug delivery device and wherein the information is selected from the group comprising of drug types a user may receive, drug types a user may not receive, a maximum dose for a medicament, a minimum dose for a medicament, a required dose for a medicament, and a dosing speed for a medicament.

28. The drug delivery system of claim 25, wherein the drug delivery device provides information to the RFID device and wherein the information is selected from the group comprising of a medicament held in the drug delivery device, a number of doses dispelled from the device, and a remaining amount of a medicament in the device.
